(19) United States
(12) Patent Application Publication
(10) Pub. No.: US 2024/0225469 A9
(43) Pub. Date: Jul. 11, 2024

(54) Methods And Devices For Assessing And Modifying Physiologic Status Via The Interstitial Space

(71) Applicant: NXT Biomedical, LLC, Irvine, CA (US)

(72) Inventors: Robert S. Schwartz, Inver Grove Heights, MN (US); Glen Rabito, Lake Forest, CA (US); Joseph Passman, Costa Mesa, CA (US); Stanton J. Rowe, Newport Coast, CA (US); Abubaker Khalifa, Milton (CA)

(73) Assignee: NXT Biomedical, LLC, Irvine, CA (US)

(21) Appl. No.: 18/261,453

(22) PCT Filed: Jan. 13, 2022
(86) PCT No.: PCT/US2022/070186

§ 371 Date: Jul. 13, 2023

Related U.S. Application Data
(60) Provisional application No. 63/138,227, filed on Jan. 15, 2021.
(60) Provisional application No. 63/138,734, filed on Jan. 18, 2021.
(60) Provisional application No. 63/209,844, filed on Jun. 11, 2021.

(51) Int. Cl. A61B 5/03; A61B 5/00

(52) U.S. Cl. CPC A61B 5/036; A61B 5/6801; A61B 5/6848; A61B 5/7225; A61B 2562/0247

(57) ABSTRACT

Method and devices for treating a physiologic condition of a patient using measurements of pressure of an interstitial space of a patient as an indicator of required therapy.

### RELATED APPLICATIONS

This application claims benefit of and priority to U.S. Provisional Application Ser. No. 63/138,227 filed Jan. 15, 2021 entitled Methods and Devices for Assessing and Modifying Physiologic Status Via the Interstitial Space; U.S. Provisional Application Ser. No. 63/138,734 filed Jan. 18, 2021 entitled Methods and Devices for Assessing and Modifying Physiologic Status Via the Interstitial Space; and U.S. Provisional Application Ser. No. 63/209,844 filed Jun. 11, 2021 entitled Methods and Devices for Assessing and Modifying Physiologic Status Via the Interstitial Space; all of which are hereby incorporated herein by reference in their entireties.

### BACKGROUND

The human body is mostly composed of water and dissolved solutes. Fluid is distributed between the intracellular (within cells) compartments and extracellular (within tissue but not in cells) compartments. Additionally, fluid can be “third-spaced” which is accumulations within body cavities such as the peritoneum and pleural cavities. Some of this solution is located within cells and can be termed intracellular fluid. Some of this solution is located outside cells and can be termed extracellular fluid. Extracellular fluid is further segmented into intravascular, interstitial, and lymphatic fluid. Typically, about ⅔ of total body water is intracellular fluid and ⅓ is extracellular fluid. A quarter of the extracellular fluid is in the intravascular space.′ The proportionately dominant remainder of the extracellular fluid is comprised of the interstitial space and the lymphatic circulation.

Extracellular fluid is dispersed in the intravascular, interstitial, and lymphatic spaces. The circulatory system of a human mainly consists of the cardiovascular system and the lymphatic system. The cardiovascular system is a closed, high-pressure circulatory system with the heart acting as a central pump. The lymphatic system is an open, low-pressure circulatory system with no central pump. The interstitial compartment acts as an intermediary between the cardiovascular compartment and the lymphatic circulation.^{2,3} A visualization of the interactional between the microvascular bed, the interstitial space, and the lymphatics is shown in FIG. 1 where the lymphatics L collect fluid from the interstitial space. Fluid, solutes, proteins, lipids, etc. exchange between the microvascular circulation and the interstitial space. The Figure is from Titze, Kidney International 2013,⁴ which is hereby incorporated by reference.

Extracellular fluid transport generally works as follows: (1) Several liters of fluids are filtered via the semi-permeable membrane of the capillaries into the interstitial space every day, governed by the Starling equation as known to those of skill in the art; and, (2) The lymphatic system collects the filtered fluid that accumulates in the interstitial space (mainly water, salts, plasma proteins) and returns it to the central venous component of the cardiovascular circulation.

The interstitial compartment has at times been considered a static and relatively uninteresting space from a pathophysiologic perspective. Recent research has revealed that the interstitial space plays an important role in volume regulation (e.g., pathogenesis of congestion); is key mediator in the pathogenesis of inflammation and shock;⁵ regulates immune function; and can play a role in cancer metastasis/therapy.⁶

To illustrate utility of the methods and devices in this application, a synopsis of various disease states will be discussed with attention paid to the contributions of the interstitial space towards each pathology:

### General Congestion (Fluid Overload/Edema)

To prevent interstitial edema, return of filtered lymph fluid occurs at a rate equal to the rate of the fluid production/accumulation in the interstitial space.⁷ An increase in the amount of the filtered fluid can lead to interstitial edema with clinical manifestations such as extremity edema, pulmonary edema, dyspnea, and reduced kidney function.

### Heart Failure

The interstitial space and lymphatic system are commonly ignored in the pathophysiology of heart failure, yet their contributions to the observed clinical manifestations of heart failure are important.

Heart failure is traditionally associated with venous congestion.⁸ Increased central venous pressure (CVP) is the largest determinant of adverse clinical outcomes (e.g., impaired renal function) and is an independent predictor of mortality in patients with heart failure.⁹ Additionally, most heart failure hospitalizations are related to manifestations of venous congestion rather than low cardiac output.^{19,11}

Furthermore, congestion can promulgate further pathology. The vascular endothelial cells sense biomechanical forces, and increased hydrostatic pressure can cause the vascular endothelial cells to switch from a dormant state to an activated state, which is marked by inflammation, vasoconstriction, and an increase in the oxidative stress.¹² Therefore, a chronic state of venous congestion can lead to organ damage, such as pulmonary vascular remodeling, hepatic injury, and renal injury.

Parallel to the venous congestion, interstitial and lymphatic congestion is another hallmark of heart failure and plays a pivotal role in the symptomatic manifestation and detrimental outcomes of heart failure. In heart failure, it is likely that many parallel mechanisms contribute to the accumulation of interstitial fluid which manifests itself clinically as lower and upper extremity edema, pulmonary edema, and hepatic congestion with subsequent ascites. It is known that interstitial fluid volume expansion is more pronounced than intravascular volume expansion in heart failure.¹³

The mechanism behind interstitial fluid accumulation in heart failure may be postulated as follows:
a) Increased capillary hydrostatic pressure drives increased filtrate to flow into the interstitial spaces via Starling-governed mechanics as known to those skilled in the art.
b) Increased central venous pressure impedes lymphatic return via the thoracic duct via Hagen-Poiseuille mechanics 14 and possibly restrictive pathology at the lympho-venous junction(s).¹⁵
c) Increases in vascular permeability, irrespective of the underlying etiology (e.g., sepsis, heart failure, cancer) can cause large proteins and other solutes, such as sodium, to leak into the extravascular space.^{18,17} The increased concentration of solute and large molecules in the interstitial space elevates oncotic pressure in the interstitial space, leading to further fluid accumulation in the interstitial space, according to Starling mechanics.

All these events, occurring in parallel, lead to a positive feedback cycle that overwhelms homeostasis and leads to a heart failure decompensation, with clinical symptoms such as renal failure, dyspnea, extremity edema, and pulmonary edema as shown in FIG. 2 which shows the positive feedback cycle of congestion leading to decompensation. Since only 30-40% of total blood volume resides in the cardiovascular circulation,¹⁸ it is possible that significant volume expansion in the interstitial space is an early indicator and potentially more sensitive indicator of congestion than cardiovascular pressures. See Miller, Circ: Heart Failure 2016.¹⁹

Diuretics are the most common method used to manage volume overload in the setting of heart failure.²⁹ A few things make the titration of diuretic therapy for heart failure patients difficult in practice. These are listed below:²¹
a) Determination of euvolemic status is difficult, with many patients being discharged with residual clinical congestion (e.g., 15% of patients discharged with clinical congestion in the DOSE-AHF study²²).
b) Routine measures of congestion can only be done in the clinic, limiting the amount of touch points to titrate therapy.
c) Many patients exhibit resistance to loop diuretics (e.g., 35% of patients in the PRAISE study displayed resistance to daily furosemide dose >80 mg).²³

The listed challenges are widely known. For example, 57% of physician questions about advanced heart failure management relate to diuretic titration.²⁴ To adequately manage heart failure, it has been shown that early intervention in the “congestion cascade” with medical intervention can prevent heart failure admissions.²⁵

For example, CardioMEMs technology which is commercially offered by the cardiovascular division of Abbott is an implantable hemodynamic monitor that can be used to assess a surrogate of clinically accepted measures of congestion. In the CHAMPION trial of CardioMEMs, heart failure hospitalizations were statistically reduced compared to controls. This may have been due to practitioners receiving enabling information that resulted in diuretic changes occurring twice as often in the treatment group.²⁶

While effective, CardioMEMS is a costly, invasive implant that carries risks including bleeding, pulmonary embolism, device embolization, and arrythmias, among others.²⁷ Other systems are being developed to non-invasively monitor volume status. One example is the REDs technology created by Sensible Medical Innovations, Ltd. This technology comprises a vest that can be used to assess a surrogate of fluid in the lungs. A study of N=50 patients showed a reduction in heart failure readmissions.²⁸ However, the REDs technology provides a surrogate index that lacks a true physiologic correlate.

As is evident, there is a need for a non-invasive, easily placed technology that provides a clear metric for when to medically intervene in the heart failure congestion cascade.

### Sepsis

A hallmark of septic shock is an inflammation-mediated increase in microvascular permeability (e.g., capillary leakage), intravascular hypovolemia, and increased cardiac output.²⁹ Fluid resuscitation is required to treat hypotension and systemic inflammatory response. As shown in FIG. 3,30 in sepsis, inflammation-mediated increases in capillary permeability cause extravasation of fluid into the interstitial space. Fluid therapy (resuscitation) and pressor administration is a therapeutic maintain used to treat hypotension. This often results in fluid overload—which is correlated to increased mortality.31 See Osterman, Crit. Care. 2015.30

However, fluid administration is part of a positive feedback cycle where fluid overload is an iatrogenic consequence of this fluid therapy (e.g., 86% of patients in a retrospective study of 245 patients with sepsis had positive fluid balance during their treatment³²). To further complicate this, determining euvolemia is difficult, as mentioned above. This results in many patients being discharged with fluid overload (e.g. 35% of patients in a retrospective study of 245 patients with sepsis had fluid overload at ICU discharge³²). An important consequence of fluid overload in septic patients is an increase in mortality³¹.

Tools to guide fluid administration in septic shock have been shown to improve fluid balance, reduce the utilization of hemodialysis and reduce the utilization of mechanical ventilation³³. However, this system requires a central line and a passive leg raise to be performed by a practitioner. Furthermore, this system is expensive, requiring both capital equipment and disposable tools. Although the outcomes related to the use of this system are positive, there is a need to further simplify the process of guiding fluid resuscitation and active deresuscitation.

A tool to more reliably, easily, and non-invasively determine physiologic euvolemia in septic patients would be useful to guide fluid administration as well as fluid off-loading via medical therapies such as hemodialysis and diuretics. Fluid is administered intravenously to maintain adequate blood pressure and consequently, end organ perfusion. However, inflammation-mediated increases in capillary permeability cause extravasation of fluid into the interstitial space. For example, a study comparing septic patients to non-septic patients found higher volumes of extracellular water in septic patients.³⁴ Researchers have hypothesized that the interstitial space may provide signals to guide the management of sepsis more adequately. There is an on-going study comparing interstitial pressure in septic and non-septic patients (NCT03818269). It is possible that probing the interstitial space may prove useful in guiding the management of sepsis.

### Chronic Kidney Disease (CKD)

Kidney disease is the 9th-ranked cause of death in the United States accounting for 13% of age-adjusted deaths, as of 2017.³⁵ Declining kidney function is stratified across five stages of chronic kidney disease (CKD) by estimated glomerular filtration rate (eGFR). End-stage renal disease (ESRD) is nomenclature for Stage 5 CKD, defined³⁶ as an eGFR <15 mL/min/1.73 m². As of 2015, there are 700,000 prevalent patients with ESRD in the U.S. This U.S. ESRD population is growing at about 2,128 cases per million population per year (approximately 700,000 patients per year).³⁷ Renal replacement therapy (RRT) is used to manage ESRD patients. RRT comprises either kidney transplant, peritoneal dialysis (PD), hemodialysis (HD), or a combination thereof. As of 2020, there is a world-wide prevalent population of 2.5 million people with ESRD treated with RRT. It is estimated that 2.3-7.1 million people have died due to not receiving RRT.³⁸ The global prevalence of people receiving RRT is expected to increase to 5.4 million by 2030.³⁹

HD dominates the treatment of ESRD, accounting or ˜90% of all treatments.⁴⁹ Patients undergoing regular HD therapy for ESRD retain fluid between dialysis treatments. A major goal of each HD therapy session is to achieve a “dry weight.” Dry weight is usually defined as the lowest weight a patient can tolerate without the development of symptoms such as hypertension.⁴¹ Achieving dry weight is important because volume excess at the end of a HD session is associated with mortality.⁴²

The estimation of dry weight is clinically derived, typically using metrics such as measures of increased preload (typically atrial natriuretic peptide levels, cyclic guanidine monophosphate levels, and vena diameter on ultrasound), surrogates of extracellular fluid levels (typically bioimpedance), and blood volume monitoring. Other common methods include looking for normal blood pressure, clinical edema, jugular vein distention, absence of rales on auscultation, no dyspnea, and a normal size heart shadow on x-ray.⁴³ There is no standard method that exists, and there is a sentiment that the current methods of assessing dry weight are antiquated and imprecise.⁴⁴

There is a need for methods and tools which probe the physiologic extent of fluid overload. Most of the excess fluid in CKD is in the extracellular (i.e., interstitial space). Excess of interstitial fluid is associated with mortality in CKD patients.⁴⁵ A recent study has shown that fluid overload is associated with elevated interstitial pressure in both short and long-term edematous CKD patients (FIG. 6).⁴⁶

It is possible that measuring interstitial pressure or otherwise probing the interstitial space could provide an accurate assessment of dry weight or otherwise guide treatment in CKD patients. This view is supported from the perspective that interstitial fluid pressure has more direct physiologic implication, relative to impedance-based correlates of fluid overload.⁴⁷

### Cancer

Tumor cells exist in the interstitial space.⁴⁸

The most common routes of chemotherapy delivery are intravenous and oral. In all modalities, the cancer therapeutic is delivered to the tumor via the vascular supply. This is called systemic administration. Systemic administration of cancer therapeutics is associated with a host of morbidities. 49,5° It is commonly accepted that improving bioavailability of cancer therapeutics is a need; the most common way this is being pursued is through nanoparticles and targeting moieties.⁵¹

Tumors have long been known to have elevated interstitial pressure.⁵² This elevated interstitial pressure in the tumor microvascular environment has been hypothesized as a barrier to delivery of cancer therapies. Elevated interstitial pressure in tumors has been found in a range of carcinomas. Furthermore, the interstitial pressure increases as a function of tumor size.⁵³ It may then be inferred that late-stage cancers with larger tumors may have even higher barriers for drug delivery.

Mechanistically, the original hypothesis was that the elevated interstitial pressure was due to the absence of a defined lymphatic system in solid tumors.⁵⁴ There is a possible fallacy here. It is now widely accepted that the initial spread of cancer cells is through the lymphatic vessels.⁵⁵ This view is contemporary, as evidenced by the common procedure of sentinel lymph node biopsy for identifying the metastatic status of the remaining axillary lymph nodes in patients with breast cancer.⁵⁶

Recent reviews have shown that lowering tumor interstitial pressure—for example, by cytokine antagonists—improves drug uptake and presumably therapeutic efficacy.^{57,58} Tools that monitor and change interstitial pressure may yield substantial clinical benefit.

In summary, it is evident that improvements to the treatment of numerous physiological conditions can be achieved by obtaining data related to interstitial fluid, e.g., interstitial pressure, and then using that data to optimize treatment therapies of such physiological conditions.

### SUMMARY

The present disclosure is generally directed to procedures and devices for treating a physiologic condition of a patient based on the status of an interstitial space of a patient and particularly the level of the subcutaneous interstitial pressure (SCIP) or total tissue pressure (TTP) or both, of the interstitial space of a patient.

In one embodiment, there is a method to treat a physiologic condition of a patient whereby SCIP is measured and it is determined whether SCIP is in a normal range, the normal range being typically between −8 and −1 mmHg. If the SCIP is outside a normal range, a therapy is conducted on the patient. The SCIP is monitored and the therapy is discontinued when the SCIP is in a normal range.

In one embodiment, a device for measuring interstitial pressure includes a capsule with perforations. A pressure conduit is insertable into the capsule and a pressure sensor is connected to the pressure conduit.

In one embodiment, a device for measuring interstitial pressure includes a capsule with perforations; and a pressure sensor placed inside the capsule.

In one embodiment, a device for measuring interstitial pressure includes a capsule with perforations and a pressure conduit insertable into the capsule. It also includes an amplifier connected to the pressure conduit and a pressure sensor connected to the pressure conduit.

In one embodiment, a device for measuring interstitial pressure includes a housing and a printed circuit board and microprocessor and battery disposed in the housing. A pressure sensor is associated with the housing and the microprocessor. Also, a fluid column is associated with the pressure sensor and a flush port connected to the fluid column

In one embodiment, and implantable device for measuring interstitial pressure includes a housing and a printed circuit board and microprocessor and battery disposed in the housing. A pressure sensor is associated with the housing and the microprocessor.

In one embodiment, a device for measuring interstitial pressure includes a housing and a column extending from the housing. A perforated capsule is disposed at a distal end of the column for placement in an interstitial space. The following items may be disposed in the housing: an atmospheric chamber; a pressure sensor; a flush fluid reservoir for supplying flush fluid to the interstitial space surrounding the perforated capsule; a flush fluid connection for refilling the flush fluid reservoir; a pressure fluid reservoir connected to the pressure sensor for communicating pressure from the perforated capsule via a fluid the pressure sensor; a pressure fluid connection for refilling the pressure fluid reservoir; and a micropump for pumping flush fluid to the interstitial space around the perforated capsule

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a schematic visualization of the interactional between the microvascular bed, the interstitial space, and the lymphatics;

FIG. 2 is a schematic representation of the positive feedback cycle of congestion leading to decompensation;

FIG. 3 is a flow chart representing inflammation-mediated increases in capillary permeability which cause extravasation of fluid into the interstitial space;

FIG. 4 is representation of an interstitial space;

FIG. 5 is an embodiment of measuring interstitial pressure in accordance with present disclosure;

FIG. 6 is a graphical representation of subcutaneous interstitial pressure readings correlated with invasive cardiovascular metrics in accordance with the present disclosure;

FIG. 7 is an embodiment of measuring interstitial pressure in accordance with present disclosure;

FIG. 8 is an embodiment of measuring interstitial pressure in accordance with present disclosure;

FIGS. 9A-9B are embodiments of measuring interstitial pressure in accordance with present disclosure;

FIG. 10 is a schematic representation of measuring interstitial pressure in accordance with present disclosure;

FIG. 11 is an embodiment of measuring interstitial pressure in accordance with present disclosure;

FIG. 12 is an embodiment of measuring interstitial pressure in accordance with present disclosure;

FIG. 13 is an embodiment of measuring interstitial pressure in accordance with present disclosure;

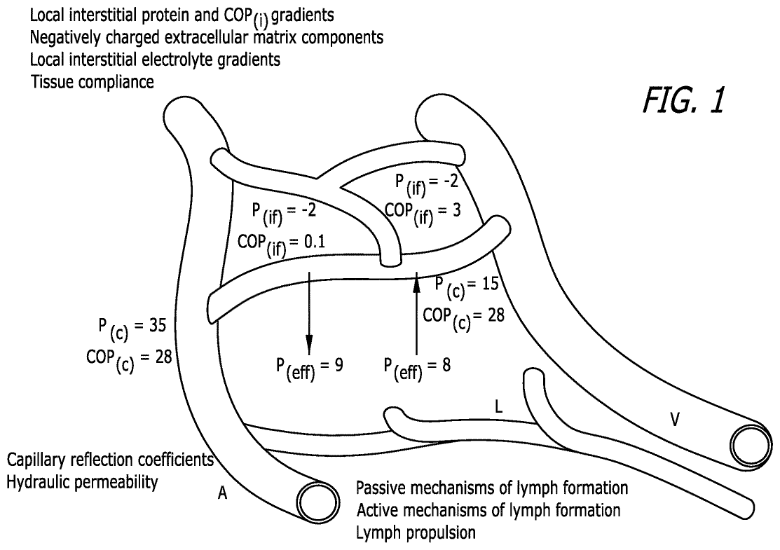

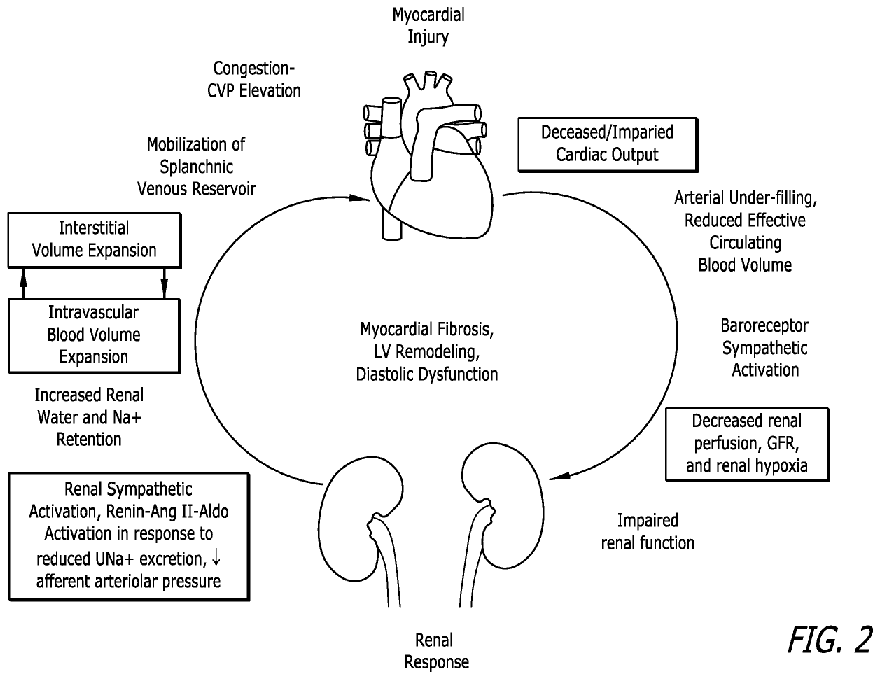

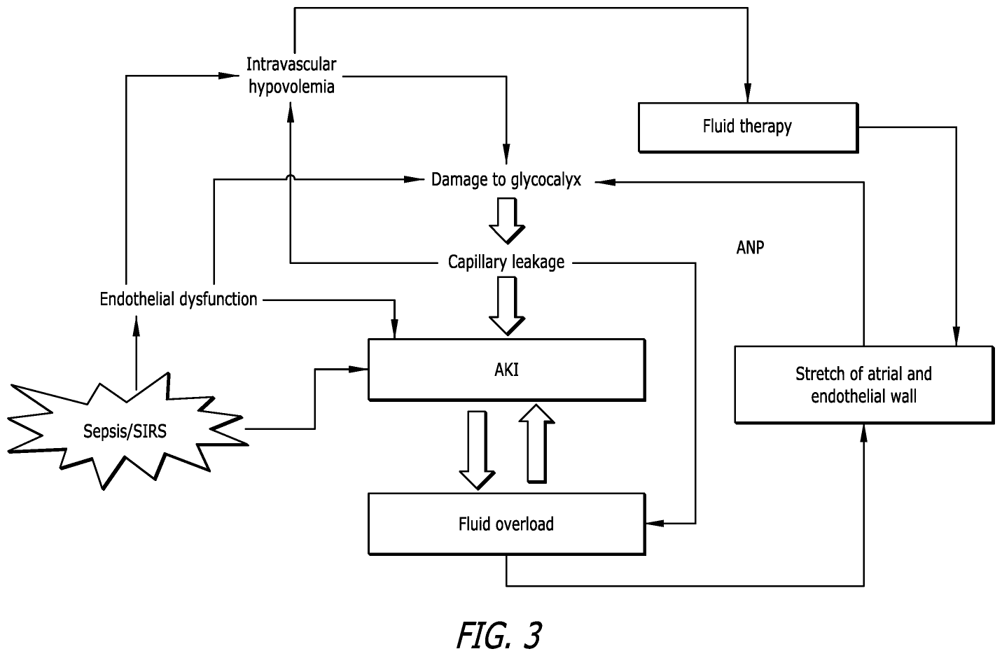

FIG. 8 is an embodiment of measuring interstitial pressure in accordance with present disclosure;

FIGS. 15A-15B are embodiments of measuring interstitial pressure in accordance with present disclosure;

FIGS. 16A-16B are embodiments of measuring interstitial pressure in accordance with present disclosure;

FIG. 17 is an embodiment of measuring interstitial pressure in accordance with present disclosure;

FIG. 18 is an embodiment of measuring interstitial pressure in accordance with present disclosure;

FIG. 19 is an embodiment of measuring interstitial pressure in accordance with present disclosure; and,

FIG. 20 is an embodiment of measuring interstitial pressure in accordance with present disclosure

### DETAILED DESCRIPTION

Specific embodiments of the disclosure will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

### General Method of Measuring Interstitial Pressure

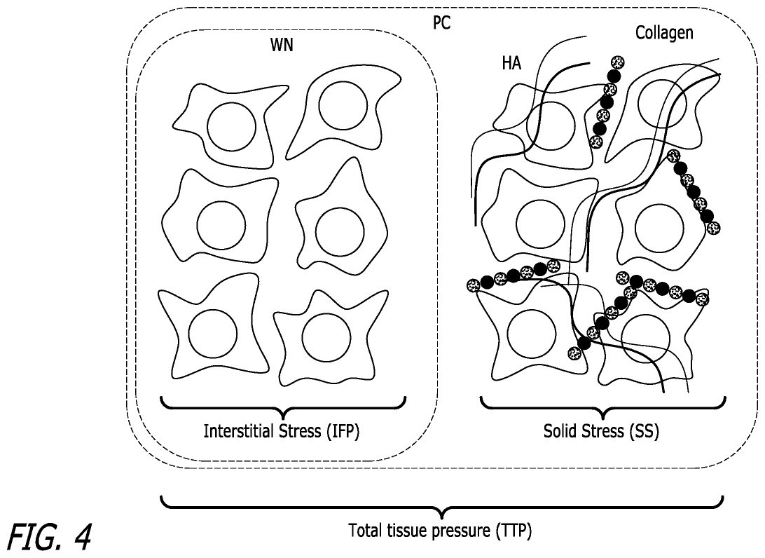

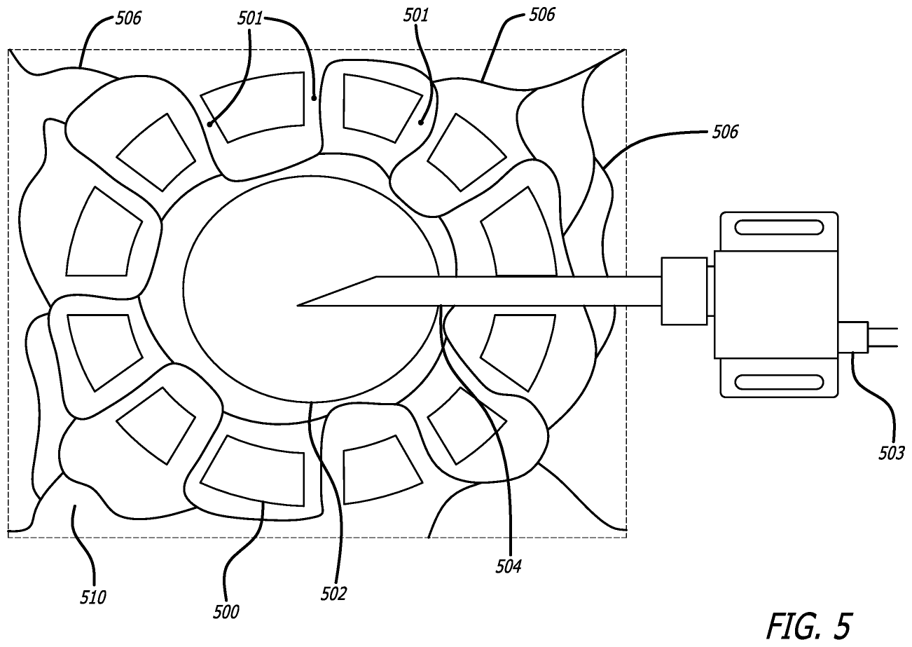

Referring to FIG. 4, the interstitial space typically exists in a gel state. As such, it will have pressure resulting from the presence of solid tissue (e.g., Types I and III collagen fibers, elastic fibers, microfibrils, and glycosaminoglycans) known as Solid Stress (SS) pressure as well as pressure resulting from the presence of fluid, known as Interstitial Fluid Pressure (IFP) or Subcutaneous Interstitial Pressure (SCIP). For the purposes of this disclosure, fluid pressure in the interstitial space shall now be referred to as SCIP.

SCIP is a primary indicator of interstitial fluid accumulation which accumulation is driven by the Starling relationship mentioned above. An equation defining the Starling relationship is set forth below where J(F) is the net flow rate at the microvascular level, L(p)A is the filtration coefficient, Pv is the pressure in the capillary bed, Pi is SCIP, a is the aggregate reflection coefficient, πᵥ is the oncotic pressure in the plasma, and πᵢ is the oncotic pressure in the interstitial space. A positive J(F) indicates that a net flux of fluid will move towards the interstitial space and could lead to edema in certain conditions.

J_{F}=LₚA[(Pᵥ−pᵢ)−σ(πᵥ−πᵢ)]

Total Tissue Pressure (TTP) is the sum of the SS pressure and SCIP.

Measuring SCIP, referred to herein as Method A, and measuring TTP, referred herein as Method B, may each be used to achieve the therapeutic improvements according to the present disclosure. Method A typically involves the use of indirect methodologies such as wicking or a perforated capsule (discussed below) to measure the amount of fluid accumulation, independent of the presence of SS.

Method B typically involves the use of an in-situ sensor such as piezoresistive or fiber optic sensor. These sensors are placed directly in the tissue, outside of the vascular space and are not mechanically shielded from the tissue. Such a sensor is sensitive to solid stress indicators resulting from such things as muscular contractions and tissue-to-tissue variations not otherwise measured with the methodologies of Method A. The measurement of such solid stress indicators are useful for evaluating fluid accumulation as well as indicating the status of the interstitial space, such as compartment syndrome.

In one embodiment both SCIP and TTP are measured, SCIP by an indirect methodology such as a perforated capsule disclosed herein and TTP by a direct methodology such as an in situ sensor disclosed herein. The measurement of both SCIP and TTP provide useful information indicating the state of the interstitial space of a patient and thus the physiologic condition of a patient as will be appreciated by one of skill in the art.

### Measuring Interstitial Pressure—Transducers

### Perforated Capsule

In one embodiment, SCIP may be measured using a perforated capsule constructed from a biocompatible polymer, such as polyurethane. Referring to FIG. 5, the capsule 500 has a diameter in the range of 1.4-2.0 cm and is perforated with holes 501. The holes 501 may be sized in a range of 0.2-1 mm. The capsule 500 may be formulated of a bio-inert polymer. Also provided is a pressure sensor 503 for use with a fluid fillable needle 504.

In one exemplary use, the perforated capsule 500 is implanted subcutaneously in, for example, a thorax, of a patient for a period of time. In one embodiment, the period may be 3-5 weeks.

During this time, interstitial tissue 510 and capillary beds 506 grow into the perforated capsule 500 through the holes 501 and form a free fluid pocket 502 inside the capsule 500.

A conduit, such as a fluid fillable needle 504 is connected to the pressure sensor 503 and the sensor is leveled or calibrated to match the level of the capsule 500. The fluid fillable needle 504 is inserted through one of the holes 501 of the capsule 500 into the free fluid pocket 502. The pressure sensor then provides signals that represent the SCIP.

In one embodiment, the time of implantation is chosen based on the diameter of the capsule 500. A smaller diameter capsule 500 may have a reduced implantation time compared to a larger diameter capsule 500. The size of the capsule 500 and time of implantation, is, in part, derived based on the speed of ingrowth of the interstitial tissue, etc. A period of time allowing too much ingrowth may diminish the formation of a free fluid pocket 502.

In one embodiment, the perforated capsule has a diameter of 2.0 cm and is implanted for a period of 3 weeks and yields a free fluid pocket 502 suitable for receiving the fluid fillable needle 504 and thus for measuring SCIP with the pressure sensor 503.

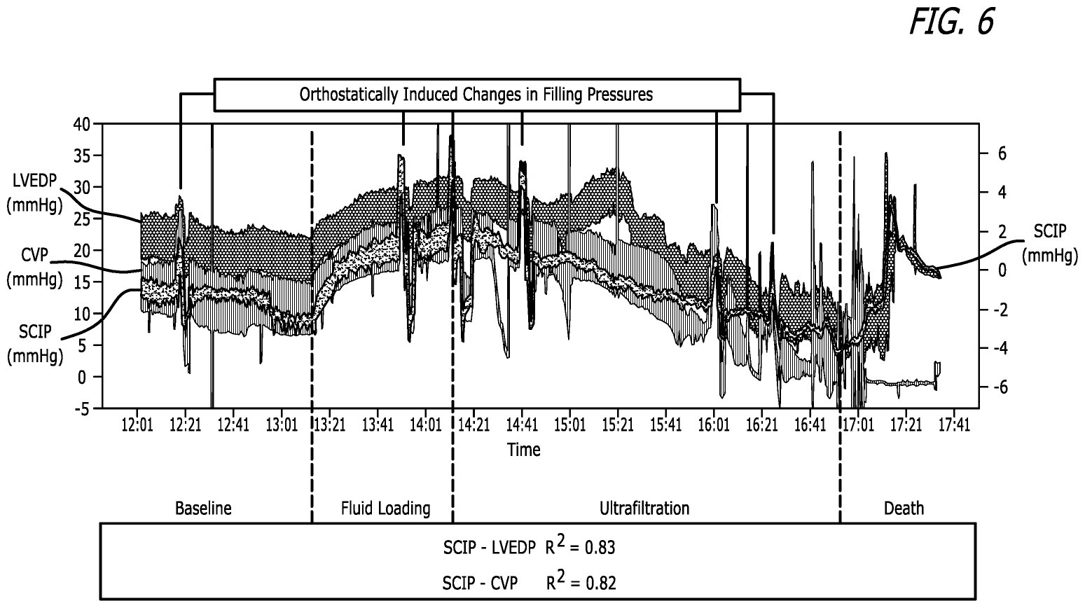

Referring to FIG. 6, a SCIP reading was made alongside two cardiovascular metrics of congestion—left ventricular end diastolic pressure (LVEDP) and central venous pressure (CVP)— in an animal model of fluid-overloaded heart failure. The heart failure model was created using a cardio-selective beta-blocker and acute fluid loading with saline. After fluid loading, congestion was alleviated via ultrafiltration of the plasma. In this manner, both fluid onloading and fluid offloading are simulated. The SCIP reading was made using an implanted perforated capsule similar to the perforated capsule embodiments disclosed herein. Furthermore, acute changes in filling pressures were made using orthostatic maneuvers (e.g., table tilting to change the position of the dependent anatomy relative to the heart). The objective of the experiment was to determine how SCIP changed relative to standard, more invasive metrics of congestion.

One hypothesis of the experiment was that SCIP would mimic changes in the vascular compartment because of microvascular exchange governed by Starling dynamics. An additional hypothesis was that time delays would exist between a rise in the vascular metrics of congestion and a corresponding rise in SCIP. Similarly, it was predicted that SCIP would lag cardiovascular metrics of congestion during the fluid offloading phase of the experiment.

Referring to FIG. 6, the raw LVEDP, CVP, and SCIP traces are shown. The peak-to-peak amplitude (vertical axis) change or vertical width of the of the CVP and LVEDP traces can be accounted for by respiratory motion. Less corresponding respiratory motion (i.e., smaller amplitude than CVP and LVEDP) can be seen in the SCIP trace. This is intuitive because the SCIP signal was measured via a perforated capsule on the leg.

Approximately one hour of baseline was acquired. One orthostatic maneuver was performed at baseline. After this, congestion was induced via fluid loading. LVEDP and CVP rose by approximately 10 mmHg. SCIP rose by approximately 5 mmHg. The values of all traces fell by similar magnitude during the fluid offloading portion, i.e., the ultrafiltration portion of the experiment.

The raw, unadjusted squared correlation coefficient of SCIP to LVEDP and SCIP to CVP was >0.8, showing a correlation between SCIP and the vascular metrics of congestion. A very small time delay of approximately 10 minutes was seen between the metrics of cardiovascular congestion and SCIP. This delay is unobservable at the scale of this graph. The inventors have developed an algorithm to account for this time delay while assessing the correlation of these signals. Another observation was that the change in SCIP induced by orthostatic maneuvers changed as a function of fluid load while the changes in the vascular metrics of congestion did not behave this way. This could provide a way to assess the relative severity of the interstitial fluid overload. For example, a large orthostatic change could suggest that the patient has lost interstitial compliance and has moved towards a severely fluid overloaded state. This inference could be used by the clinician to initiate a therapeutic regimen.

Using this data, it is the inventors' view that SCIP can be used to track cardiovascular metrics of congestion, and by corollary, to other physiologic metrics of congestion, such as impedance-based interstitial volume measurements, pitting edema, jugular venous pressure palpation, inferior vena cava diameter, etc. Importantly, this measurement can be obtained non-invasively, i.e., by methods disclosed herein. In this manner, physicians may have a window into cardiovascular physiology (and other physiological conditions such as acute kidney injury, cancer, sepsis, and chronic kidney disease) on a more deployable scale and with more granularity into the area of disproportionate fluid accumulation in, for example, the vicious cycle of heart failure. In addition, the movements of a patient could be tracked to sense SCIP changes during an at-home orthostatic maneuver, such as getting out of bed in the morning. This may be deduced from the data suggestion that the changes of SCIP induced by orthostatic maneuvers correspond to fluid status. This could be used to check for noise in data, to filter data, or to make treatment decisions.

In light of the foregoing, it will be appreciated that the present disclosure provides a method and protocol for the treatment of numerous physiologic conditions of a patient using measurement of SCIP as a primary indicator of the needed therapy. Non limiting examples of such methods and protocols are set forth below.

### General Congestion

The patient is admitted with congestion. In one embodiment, aSCIP sensing system in accordance with this disclosure is attached the patient. The SCIP sensing system sends continuous SCIP data to a database. In one embodiment, the patient is asked to perform orthostatic maneuvers, for example, a passive leg raise. If SCIP is elevated beyond a normal range, the physician uses physiologic knowledge to move SCIP towards a normal range using one or more fluid offloading therapies known to those of skill in the art. Therapy is stopped when SCIP reaches a normal range.

### Heart Failure

A heart failure team or physician assesses that a patient is at high risk of recurrent hospital admission due to patient history, demographics, or some other clinical assessment. In one embodiment, the heart failure physician prescribes a SCIP sensing system in accordance with this disclosure to the patient. The SCIP system is attached to the patient. The physician prescribes a patient a diuretic dosage appropriate for the patient. The SCIP sensing system sends continuous SCIP data to a database. The heart failure team observes the data and notes any abnormalities in SCIP. Several orthostatic changes naturally occur during the observation window. In one embodiment, upon observation of an abnormality in SCIP (e.g., elevation in SCIP) changes in medical therapy, e.g., diuretic dosage, are determined in order to bring SCIP value into normal range, i.e., to remove the abnormality. In one embodiment, the recommended changes will be reported to the patient. Changes in the medication prescription are logged, and the impact on SCIP is observed. Treatment changes are determined to be adequate when SCIP moves towards a normal range. The above is an example of an intervention that can be used to prevent hospitalization due to fluid overload and associated dyspnea.

### Sepsis

Sepsis is associated with rapid onset interstitial fluid loading due to increased capillary permeability. One therapy is to administer fluid to counteract the massive loss of intravascular fluid to the interstitial compartment. A main guiding signal is arterial blood pressure (ABP). It is of importance to the intensivist that the ABP does not drop. Therefore, it is not uncommon that an intensivist will over-administer fluid and induce iatrogenic fluid overload. Iatrogenic fluid overload in sepsis is associated with increased mortality. The SCIP sensing system in accordance with this disclosure is placed on a patient upon admission for sepsis. In one embodiment, the SCIP signal is monitored alongside the ABP signal to obtain a quantitative understanding of the fluid distribution, answering how the vascular and interstitial compartments are accumulating fluid relative to one another. In one embodiment, this information is used by the intensivist to determine when to stop fluid loading. In one embodiment, the SCIP signal is used to guide active deresuscitation via ultrafiltration, continuous renal replacement therapy, etc. In one embodiment, the physician uses the SCIP signal to determine the rate at which to offload fluid and to determine the total volume to remove. In one embodiment, the SCIP signal is used by the clinician through understanding normal physiologic ranges or by a predictive analytic algorithm trained on preventing morbid events in sepsis.

### Kidney Disease

Kidney disease is a prevalent condition with fluid overload that occurs between every dialysis session. A principal goal of dialysis is moving a patient safely to dry weight. To do this, a nephrologist must determine how much fluid to remove and the rate of fluid removal. If the dialysis machine removes the fluid too quickly, the vascular space may be depleted and cause intradialytic hypotension. In one embodiment, a SCIP sensing system in accordance with this disclosure is used to inform the rate of fluid removal. In one embodiment, a SCIP sensing system in accordance with this disclosure is attached to a patient. In addition, Arterial Blood Pressure is monitored. If Arterial Blood Pressure, ABP, is dropping but the SCIP is not changing, it is possible the ultrafiltration rate is too high. Accordingly, ultrafiltration (fluid offload) is reduced. In addition, dry weight determination can be difficult. Usually, physicians simply offload fluid until the patient has cramps or other intradialytic morbid event to determine dry weight. This is imprecise and fails to use quantitative data on the primary location of fluid overload to inform how much fluid to offload. The SCIP sensing system provides the fluid overload information and a physician can adjust the treatment/therapy accordingly.

### Cancer

Cancer cells are typically located in the interstitial space. In other words, they sit “outside” normal cells. The growth of cancer cells crowds the interstitial compartment and increases interstitial fluid pressure. Low interstitial fluid pressure is essential to drive chemicals such as cancer treatment therapies into the interstitial space, as governed by Starling dynamics and importantly, the difference between capillary pressure and interstitial fluid pressure. In cancer, capillary pressures can be normal and interstitial fluid pressure elevated. This acts as a hydrodynamic barrier to therapeutic secretion of cancer treatment into the interstitial space. The flux of therapeutic to the cancer cells, through the interstitial space, is critical to increasing the bioavailability of therapeutic to the cancer cell. In one embodiment, prior to an intravenous therapeutic intervention of cancer drugs, a SCIP sensing system in accordance with this disclosure is placed on a patient. In one embodiment, if elevated SCIP is observed, a physician prescribes a therapeutic, such as a vasodilator or an agent that increases vascular permeability or an agent that increases net flux from the interstitial space to the vascular compartment, to lower SCIP. Once SCIP is at a normal level, the cancer therapeutic is administered. This increases the efficacy of cancer therapeutics.

### Perforated Capsule—Foam Lined

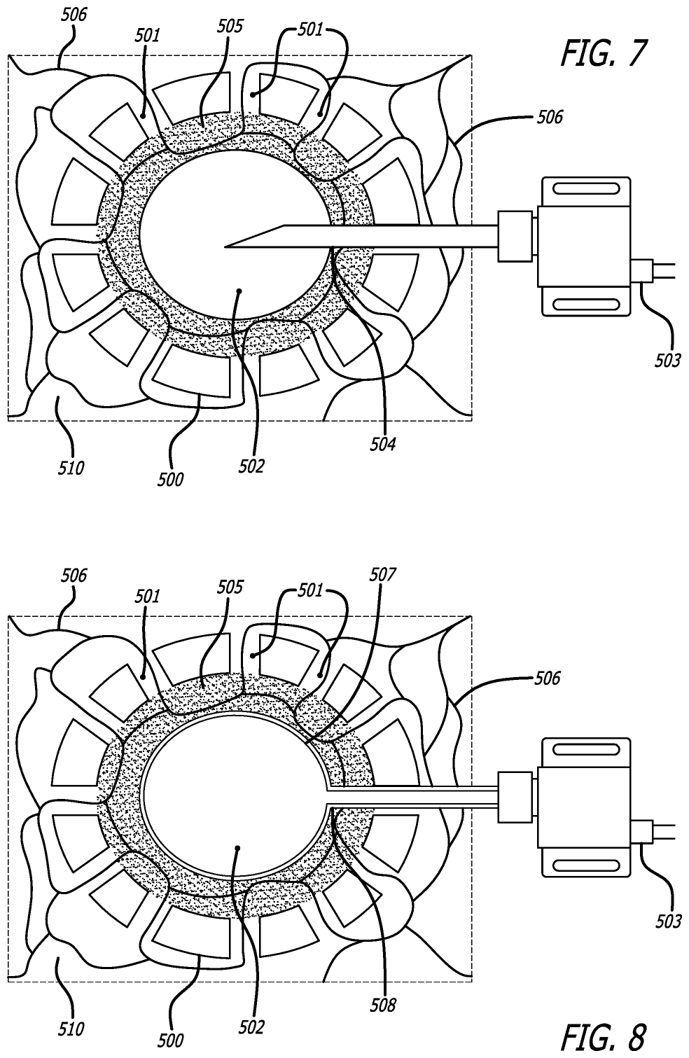

Referring to FIG. 7, a perforated capsule 500 as described above with reference to FIG. 6 is shown. However, the perforated capsule includes a foam matrix 505 lining the interior of the perforated capsule 500. The foam matrix 505 causes the ingrowth of the interstitial tissue and capillaries to occur in a uniform and predictable manner and ensures the formation of the free fluid pocket 502. Usage of the perforated capsule with the foam matrix 505 may follow the same protocol as set forth with respect to the perforated capsule of FIG. 6. The measured pressure is the SCIP.

### Perforated Capsule—Foam Lined, Balloon

Referring to FIG. 8, a perforated capsule 500 with a foam matrix 505 as described above with reference to FIG. 7 is shown. However, the perforated capsule 500 includes a balloon lining 507 adjacent the foam matrix 505. The balloon lining 507 serves as a barrier to the ingrowth of the interstitial tissue 510 and capillary beds 506 and ensures the formation of the free fluid pocket 502.

A conduit such as a fluid column 508 is either added to the balloon lining 507 or an integral part of the balloon lining 507 (molded or extruded) and is attached to the pressure sensor 503. Usage of the perforated capsule with the foam matrix 505 and the balloon lining 507 may be similar to the protocol discussed above with respect to the perforated capsule of FIG. 6. However, instead of a conduit in the form of a needle, a conduit in the form of the fluid column 508 serves as the conduit of pressure to the pressure sensor 503. The measured pressure is the SCIP

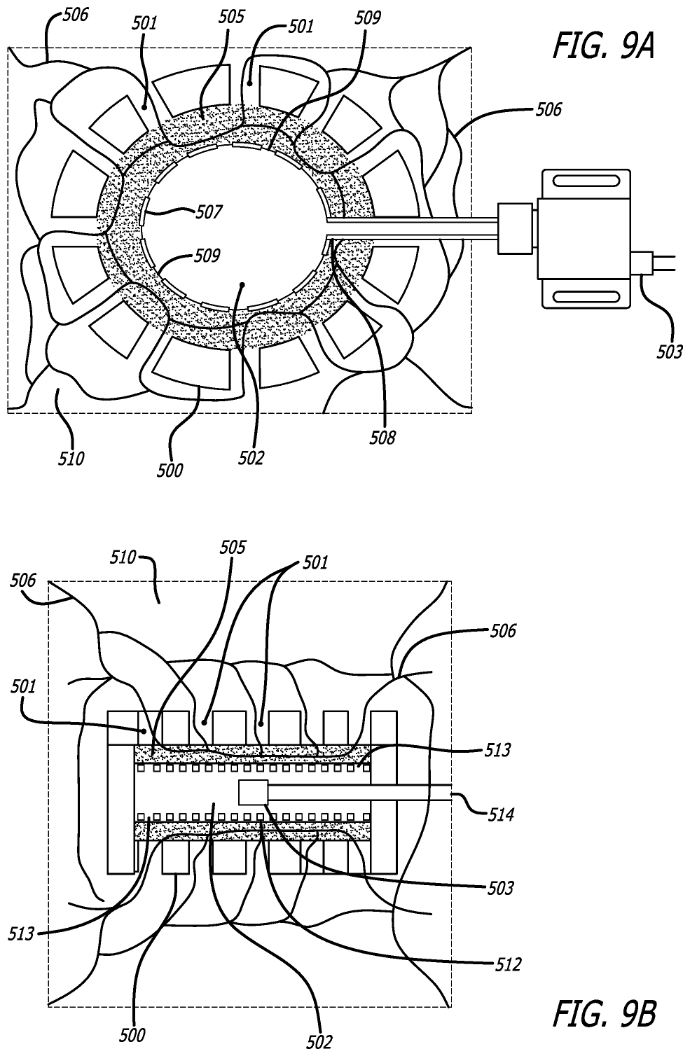

Referring to FIG. 9A, a perforated capsule 500 with a foam matrix 505 and a balloon lining 507 as described above with reference to FIG. 8 is shown. However, the perforated capsule 500 includes a balloon lining 507 with small pores 509. The balloon lining 507 serves as a barrier to the ingrowth of the interstitial tissue and capillaries and ensures the formation of the free fluid pocket 502. The small pores 509 of the balloon lining 507 allow free fluid transfer of the interstitial fluid into the free fluid pocket 502. In one embodiment the small pores are sized in a range of 5-10 microns.

A fluid column 508 is either added to the balloon lining 507 or an integral part of the balloon lining 507 (molded or extruded) and is attached to the pressure sensor 503. Usage of the perforated capsule 500 with the foam matrix 505 and the balloon lining 507 with small pores 509 may be similar to the protocol discussed above with respect to the perforated capsule of FIG. 6. However, instead of a needle, the fluid column 508 serves as the conduit of pressure to the pressure sensor 503. The measured pressure is the SCIP.

### Perforated Capsule—In Situ Sensor

Referring to FIG. 9B, a perforated capsule 500 (with a different shape than shown in FIG. 9A) with a foam matrix 505 and a lining 512 (e.g., polymer) is shown. The lining 512 with small pores 513. The lining 512 serves as a barrier to the ingrowth of the interstitial tissue 510 and capillary beds 506 and ensures the formation of the free fluid pocket 502. The small pores 513 of the lining 512 allow free fluid transfer of the interstitial fluid into the free fluid pocket 502. In one embodiment the small pores are sized in a range of 5-10 microns.

A pressure sensor 503 is located in situ, meaning in the free fluid pocket 502. The pressure sensor 503 has a connection 514 to electronics as is known to those of skill in the art. Usage of the perforated capsule 500 with the foam matrix 505 and the lining 512 with small pores 513 may be similar to the protocol discussed above with respect to the perforated capsule of FIG. 6. However, the pressure sensor 503 is located in situ. The measured pressure is the SCIP.

It will be understood by one of skill in the art that “noise” from the patient such as muscular contractions, physical movement, etc. may introduce perturbations to the SCIP signal being measured by the pressure sensor 503. Such noise may cause the SCIP to exceed the range of a normal physiologic reading for a patient and thereby give intermittent inaccurate readings. Accordingly, in one embodiment, it may be useful to amplify or intensify the SCIP signal from the pressure sensor 503 and thereby improve the signal to noise ratio so such perturbations can be mitigated.

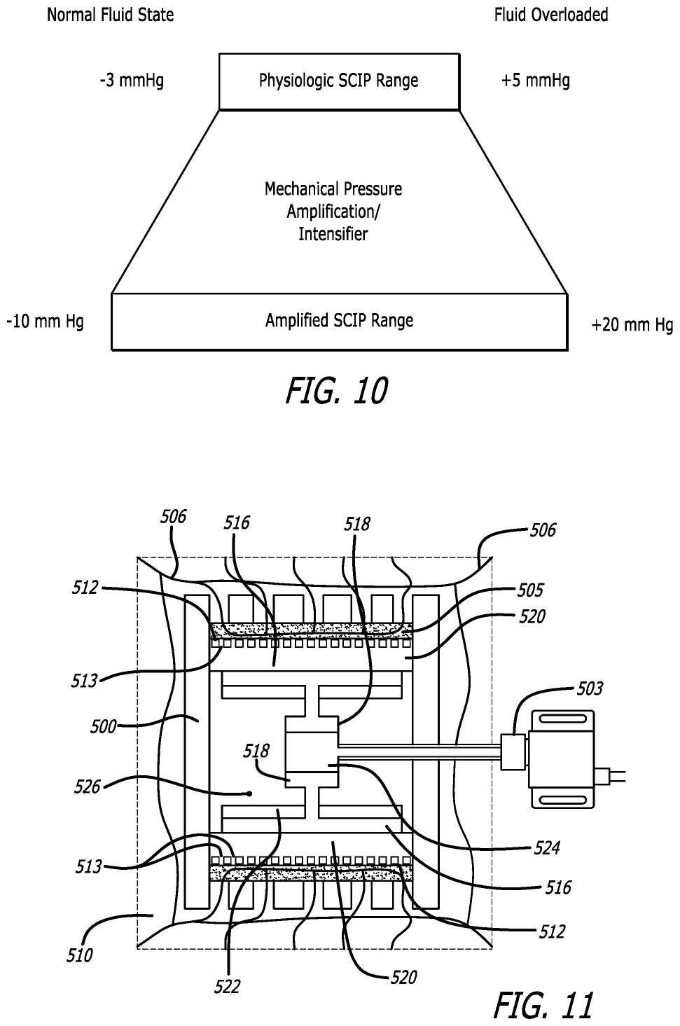

A typical SCIP is in the range of −5 to −3 mmHg. In a fluid overload state SCIP increases to approximately +2 mmHg. Referring to FIG. 10, one embodiment of a pressure amplification method is shown. For example, a pressure sensor 503 is calibrated to read SCIP within a physiologic range of −3 mmHG (normal fluid state) and +5 mmHG (fluid overloaded state). A mechanical pressure amplification/intensifier is integrated with the pressure sensor and thus leads to an amplified SCIP range of −10 mmHG (normal fluid state) to +20 mmHG (fluid overloaded state). The intensifying of the physiologic pressure changes in this manner could exist for the purposes of improving the signal to noise ratio or increase the true positive rate of detecting fluid overload.

### Perforated Capsule—Foam Lined, Amplifier

Referring to FIG. 11, one embodiment of a SCIP measuring system with a mechanical pressure amplifier is shown. In one embodiment, a perforated capsule 500 is shown. In one embodiment the capsule has a width in a range of 0.5-2.0 cm and a length in a range of 0.5-3.0 cm.

The capsule 500 is lined with a foam matrix 505 and a lining 512 (e.g., polymer) is located on an inner portion of the foam matrix 505. The lining 512 has small pores 513. In one embodiment, the small pores 513 are 3-10 microns in diameter. The lining 512 serves as a barrier to the ingrowth of the interstitial tissue 510 and capillary beds 506. The small pores 513 of the lining 512 allow free fluid transfer of the interstitial fluid into the first fluid filled chamber 520. In one embodiment the small pores are sized in a range of 3-10 microns.

There also is a first fluid chamber 520 and a second fluid chamber 524 in the perforated capsule 505 formed by a lubricious polymer body 526 positioned in the perforated capsule 505. The first fluid chamber 520 and second fluid chamber 524 are pre-filled with a non-compressible liquid (e.g., silicone oil, saline, etc.) The lining 512 may be composed of a hydrophilic polymer or it may contain a hydrophilic coating. This would prevent the silicone oil from leaving the fluid chamber 520. In other embodiments where the fluid chamber 520 is filled with saline, natural flux of water with the interstitial environment would not be impeded, and changes in true SCIP would control the flux between the interstitial compartment and the fluid chamber 520. A pressure sensor 503 is fluidly connected to the second fluid chamber 524 via a fluid column. In one embodiment, an in-situ pressure sensor is located within the second fluid chamber 524.

The first fluid chamber 520 and second fluid chamber 524 are connected to each other via a piston system that comprises a first pressure disc 516 and a second pressure disc 518. The surface area of the first pressure disc 516 is larger than the surface area of the second pressure disc 518. Accordingly, in operation, changes to SCIP as applied to the first fluid chamber 520 induce a force against the first pressure disc 516. The piston system is allowed to translate because of a sealed air pocket 522. A lubricious polymer body 526 exists as a seal for the pocket and also allows the pistons to translate, if needed, along the air pocket chamber 522 with minimal friction. The piston system thus exerts a pressure change into the second fluid chamber via the second pressure disc 518. The pressure change exerted into the second fluid chamber is amplified (i.e., is larger than the pressure change in the first fluid chamber) because the surface area of the first pressure disc 516 is larger than the surface area of the second pressure disc 518.

Usage of the perforated capsule 500 with the foam matrix 505 and the lining 512 with small pores 513 as set forth in FIG. 11 may be similar to the protocol discussed above with respect to the perforated capsule of FIG. 6. However, the pressure readings have a higher signal-to-noise ratio given the amplification of the pressure reading provided by the piston system and fluid chambers.

Protective Tube with In-Situ Sensor

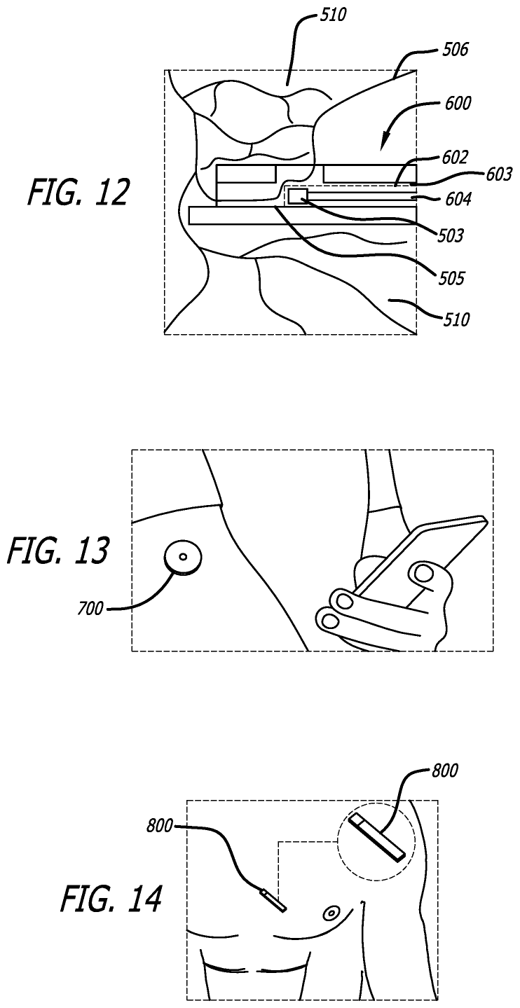

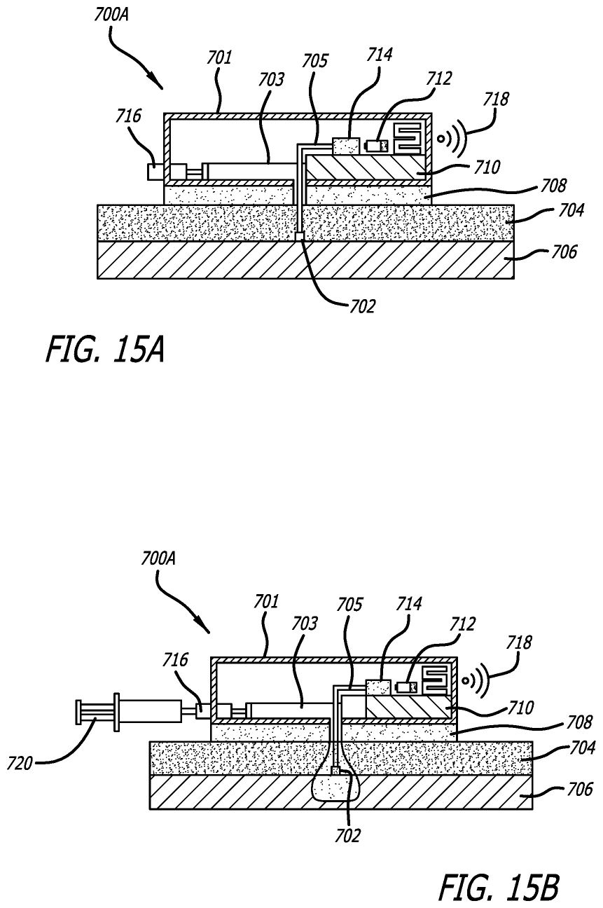

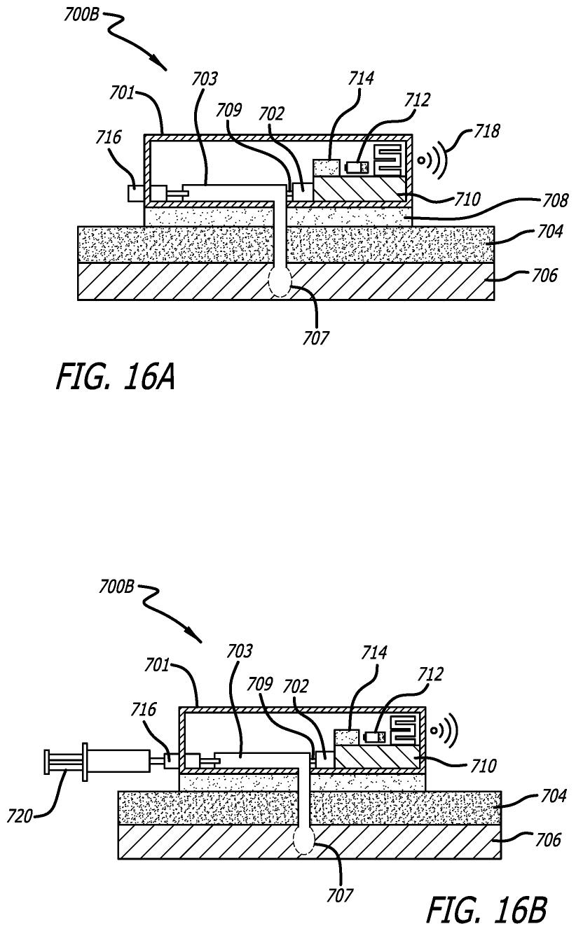

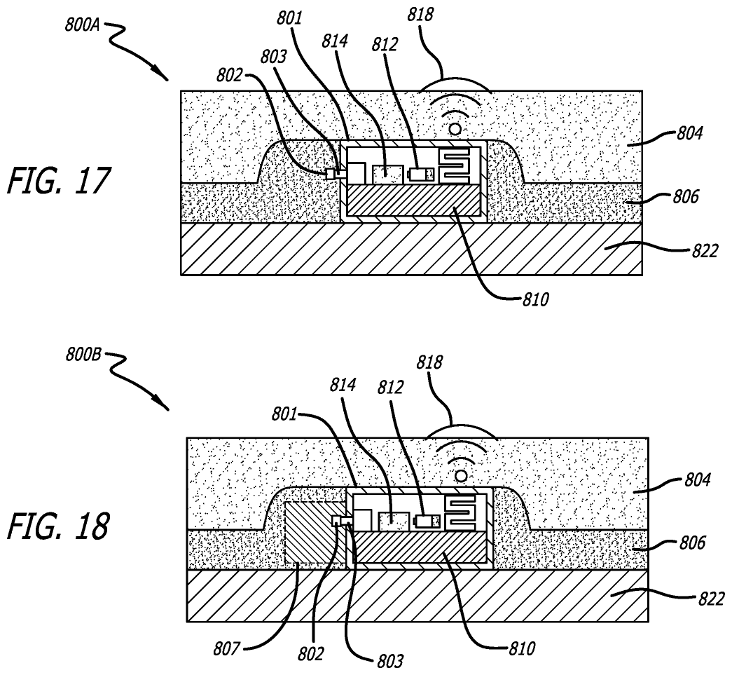

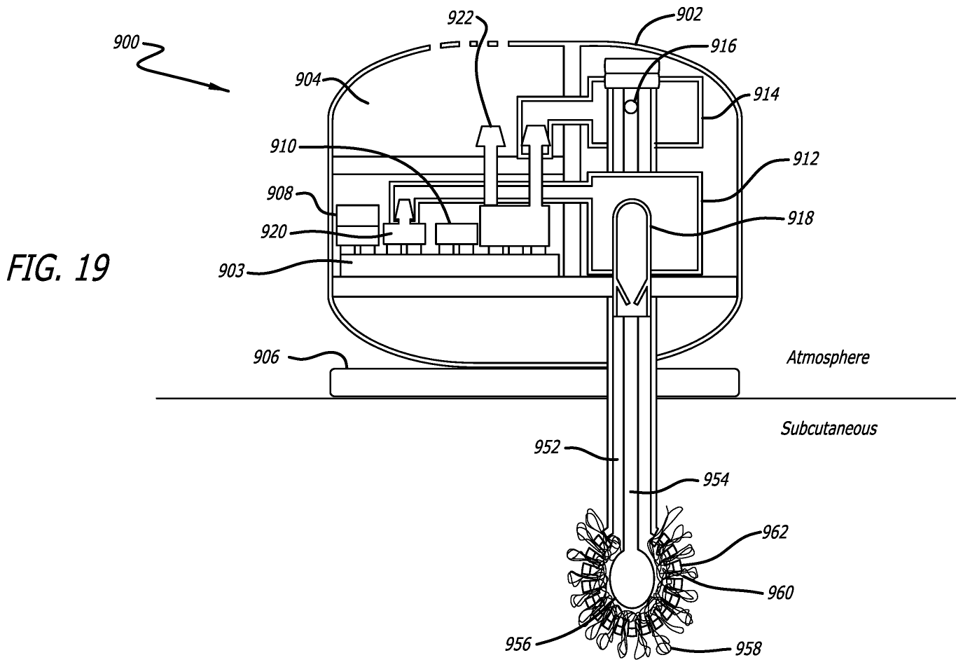

Referring to FIG. 12, a tube 600 contains an in-situ pressure sensor 503 surrounded by a foam matrix 505. In one embodiment, the foam matrix may also include a lining 602 between the foam matrix 505 and the pressure sensor 503 and has small pores 603. In one embodiment, the lining is a polymer lining and the small pores 603 have a size ranging from 6-10 microns.

In one embodiment, the in-situ pressure sensor can be a fiber optic transducer, a piezoresistive transducer or a strain-gage transducer.

The capillary bed 506 and in the interstitial tissue 510 grows into the foam matrix. The small pores 604 inhibit tissue and capillary growth into a space around the pressure sensor 503 for creating a free fluid pocket. Electronics 604 connect the pressure sensor 503 to a pressure reading device as know to those of skill in the art.

In some embodiments, the tube 600 can be a needle or a polymer tube. In some embodiments the tube 600 has a pre-filled fluid chamber (e.g., with silicone oil or water, etc.) instead of a foam matrix 505.

In some embodiments, the foam matrix 505 may be initially filed with non-compressible fluid and de-aired. This means that the foam matrix 505 may replace the function of a traditional wick-in-needle (WIN) method of measuring SCIP. The WIN method may encounter clogging induced by ingrowth or mechanical compaction of interstitial tissue. Using a bioinert foam matrix can reduce these problems. In one embodiment, it is desirable that the foam matrix 505 be free of air to make sure the pressure changes in the interstitial space are not dampened by a compressible medium like air. De-airing, for example, by immersing the transducer in saline prior to device delivery, reduces the chance of air remaining in the system after insertion. This process and design may improve the sensitivity and stability of an in-situ transducer relative to traditional methods such as WIN.

Usage of the tube 600 to measure SCIP may be similar to the protocols discussed above.

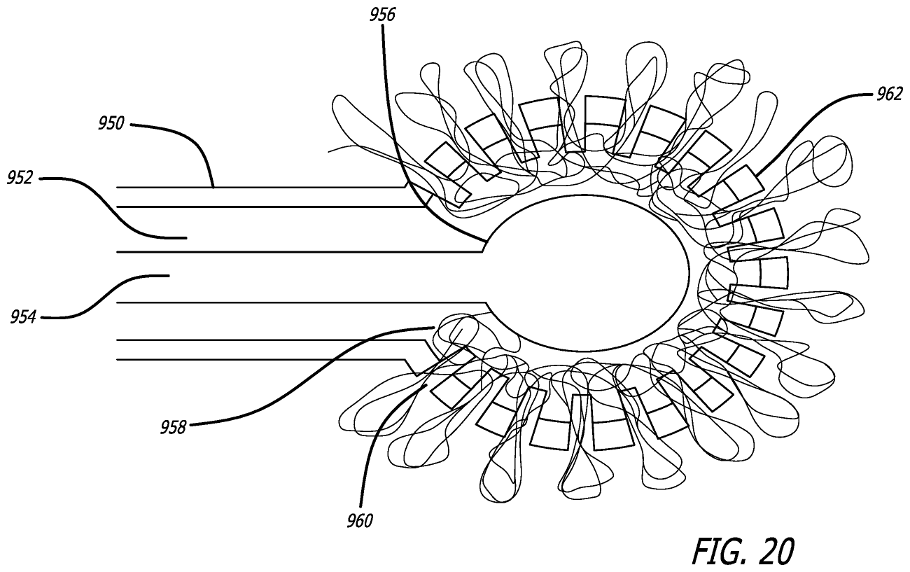

The above-discussed concepts (and others not specifically enumerated herein) can be integrated into a number of practically useful products for measuring SCIP. One product may be a wearable device 700 that can be applied to a user's arm or other location on the skin as shown in FIG. 13. Such a wearable device 700 could be periodically replaced, e.g., every 14 days; can be applied by a user (e.g., a patient); can be controlled and/or monitored remotely, e.g., through a mobile phone; and can also integrate other physiologic measurements such as heart rate, ECG, bioimpedance, PPG, blood pressure, etc.

Another such product may be a subcutaneous implant 800 that can be implanted beneath a patient's skin, e.g., in a patient's chest, as shown in FIG. 14. Such an implant 800 could have an extended life, e.g., 5 years and could also integrate other physiologic measurements such as heart rate, ECG, bioimpedance, PPG, blood pressure, etc.

Wearable Device with In Situ Pressure Sensor

Referring to FIGS. 15A and 15B, a wearable device 700A is shown having a housing 701 which houses components known in the art to process signals such as a pressure sensor signal and to wireless communicate information to a user. In one embodiment such components include a printed circuit board (PCB) 710, a battery 712, an ND converter and microcontroller unit (MCU) 714, a blue tooth device 718. Also in the housing is a fluid column 703 and a flush port 716. An electronics extension 705 extends outside of the housing 701 and is attached to a pressure sensor 702.

The housing is adhered to a user's skin with an adhesive 708 and, in doing so, the sensor 702 is located either in the epidermis 704 or the deep epidermis 706 of the user or in a range between the two. In this fashion the sensor 702 is then deemed an in situ sensor. As such, the sensor 702 is positioned to read TTP as pressure is measured directly and not indirectly through, for example, a perforated capsule.

In one embodiment, the wearable device can have both an in-situ sensor as described as well as a sensor housed in, for example, a perforated capsule for measuring SCIP. The device then is capable of measuring both TTP and SCIP, which, in one embodiment provides further useful information as to the physiologic condition of a patient as will be appreciated by one of skill in the art.

Referring to FIG. 15B, a syringe 720 or other plunger mechanism is attached to the flush port 716 and saline or other suitable fluid is urged into the fluid column 703 to flush the area in the epidermis 704, 706 around the sensor 702. The flush ensures a space around the sensor 702 to allow accurate pressure readings.

In one embodiment, the pressure sensor 702 communicates signals representative of SCIP to the ND converter and MCU 714. An algorithm as is known to those of skill in the art is used by the MCU to process the pressure into pressure signals that are then communicated via the blue tooth device 718 to a receiving device such as a mobile phone.

In one embodiment, the wearable device 700A is a patch.

Wearable Device with Perforated Capsule

Referring to FIGS. 16A and 16B, a wearable device 700B is shown having a housing 701 which houses components known in the art to process signals such as a pressure sensor signal and to wirelessly communicate information to a user. In one embodiment such components include a printed circuit board (PCB) 710, a battery 712, an ND converter and microcontroller unit (MCU) 714, a blue tooth device 718. Also in the housing is a fluid column 703 and a flush port 716. A pressure column 709 connects the fluid column 703 to a pressure sensor 702. The fluid column 703 extends outside of the housing 701 and is attached to a perforated capsule 707.

In one embodiment the perforated capsule 707 can be a perforated capsule as discussed above with respect to other embodiments. In one embodiment the perforated capsule 707 may be a Guyton capsule as is known to those of skill in the art.

The housing is adhered to a user's skin with an adhesive 708 and, in doing so, the perforated capsule 707 is located either in the epidermis 704 or the deep epidermis 706 of the user or in a range between the two.

Referring to FIG. 16B, a syringe 720 or other plunger mechanism is attached to the flush port 716 and saline or other suitable fluid is urged into the fluid column 703 to flush the perforated capsule 707 and the surrounding area in the epidermis 704, 706. The flush ensures a space within and around the sensor perforated capsule 707 to allow accurate pressure to be communicated from a free fluid pocket of the perforated capsule 707 through the fluid column 703 to the pressure column 709 connected to the pressure sensor 702.

The pressure sensor 702 communicates signals representative of SCIP to the ND converter and MCU 714. An algorithm as is known to those of skill in the art is used by the MCU to process the pressure into pressure signals that are then communicated via the blue tooth device 718 to a receiving device such as a mobile phone.

In one embodiment, the wearable device 700B is a patch.

Implantable Device with In Situ Pressure Sensor

Referring to FIG. 17, an implantable device 800A is shown having a housing 801 which houses a printed circuit board (PCB) 810, a battery 812, an ND converter and microcontroller (MCU) 814, a blue tooth device 818. An electronics connection 803 extends outside of the housing 801 and is attached to a pressure sensor 802.

The housing 801 is implanted either in the epidermis 804 or the deep epidermis 806 or in a range including the two. In one embodiment, the housing is above the fascia 822. In doing so, the sensor 802 is located either in the epidermis 804 or the deep epidermis 806 of the user or in a range between the two. In this fashion the sensor 802 is then deemed an in situ sensor. As such, in one embodiment, the sensor 802 is positioned to read TTP as pressure is measured directly and not indirectly through, for example, a perforated capsule.

In one embodiment, the implantable device can have both an in-situ sensor as described as well as a sensor housed in, for example, a perforated capsule for measuring SCIP. The device then is capable of measuring both TTP and SCIP, which, in one embodiment provides further useful information as to the physiologic condition of a patient as will be appreciated by one of skill in the art

In operation, the pressure sensor 802 communicates signals representative of SCIP to the ND converter and MCU 714. An algorithm as is known to those of skill in the art is used by the MCU to process the pressure into pressure signals that are then communicated via the blue tooth device 718 to a receiving device such as a mobile phone.

In one embodiment, the system of FIG. 17 measures total tissue pressure.

Implantable Device with Perforated Capsule

Referring to FIG. 18, an implantable device 800B is shown having a housing 801 which houses components known in the art to process signals such as a pressure sensor signal and to wirelessly communicate information to a user. In one embodiment such components include a printed circuit board (PCB) 810, a battery 812, an ND converter and microcontroller unit (MCU) 814 and a blue tooth device 818. An electronics connection 803 extends outside of the housing 801 and is attached to a pressure sensor 802. Also attached to the housing is a perforated capsule 807.

In one embodiment the perforated capsule 807 can be a perforated capsule as discussed above with respect to other embodiments. In one embodiment the perforated capsule 807 may be a Guyton capsule as is known to those of skill in the art.

The housing 801 is implanted either in the epidermis 804 or the deep epidermis 806 or in a range including the two. In one embodiment, the housing is above the fascia 822. In doing so, the perforated capsule 807 is located either in the epidermis 804 or the deep epidermis 806 of the user or in a range between the two.

In one embodiment a pressure sensor 802 is located inside the perforated capsule 807 and is connected to the MCU 814 via an electronics connection.

In another embodiment, a pressure sensor 802 is located inside the housing 801 and a pressure column extends from the pressure sensor 802 to a free fluid compartment inside the perforated capsule 807.

In operation, the pressure sensor 802 communicates signals representative of SCIP to the ND converter and MCU 714. An algorithm as is known to those of skill in the art is used by the MCU to process the pressure into pressure signals that are then communicated via the blue tooth device 818 to a receiving device such as a mobile phone.

In one embodiment, the system of FIG. 18 measures total tissue pressure.

Attached Device with Perforated Capsule

Referring to FIG. 19, a device attachable or wearable by a user 900 is shown having a housing 902 which houses a printed circuit board (PCB) 903; a battery 908, a processing unit 910 having, for example, a blue tooth mechanism, an MCU, storage mechanism, etc.

Extending from the housing 902 and into a subcutaneous space is a column containing a perforated capsule 962 and its distal end. In one embodiment, the perforated capsule 962 can be one of any perforated capsules previously described herein.

The housing 902 also houses an atmospheric chamber 904 which has holes to the atmosphere allowing the chamber to be maintained at an atmospheric pressure; a flush fluid reservoir 912 for supplying flush fluid (e.g., saline) to an interstitial space surrounding the perforated capsule 962; a flush fluid connection 916 for refilling the flush fluid reservoir 912; a pressure fluid reservoir 914 connected to a pressure sensor 922 for communicating pressure from said perforated capsule 962 via a fluid, e.g., silicone to the pressure sensor 922; a pressure fluid connection 916 for refilling the pressure fluid reservoir; and a micropump 920 for pumping flush fluid to the interstitial space around the perforated capsule 962.

The pressure sensor 922 is a dual port sensor with one port exposed to atmosphere in the atmospheric chamber 904 and a second port connected to the pressure fluid reservoir 914

Referring to FIG. 20, the column extending from the housing 902 may be constituted by a braided cover 950 having the perforated capsule 952 connected or coextensive therewith. The perforated capsule 962 has small pores 960. In one embodiment the shaft 950 is a polymer. In another embodiment, the shaft 950 is a braided material.

The column further is constituted by a balloon shaft 954 and a coextensive balloon 956 contained within the braided cover 950. There is a flush fluid space 952 between the balloon shaft 954/balloon 956 and the internal surface of the braided cover 950. The flush fluid space 952 is connected to the flush fluid reservoir 912.

The balloon shaft 954/balloon 956 is in fluid communication with the pressure fluid reservoir 914, which, in turn, is in fluid communication with one of the ports of the pressure sensor 922.

Integrated into the perforated capsule 962 is a wick mechanism 960 to provide a wicking action around the perforated capsule 952. In one embodiment, the wick mechanism 962 is constituted by a bioinert polymer.

In operation, the attachable device 900 is secured to a user by urging the perforated capsule 962 into a subcutaneous space of the user and the housing is adhered to the user with an adhesive or adhesive-lined flexible polymer 906.

Flush fluid (e.g., saline) from the flush fluid reservoir 912 is then urged by the micropump 920 through the flush fluid space 952 and out through the pores 960 of the perforated capsule 962 into the interstitial space. This flush ensures proper exposure of the perforated capsule 962 to the interstitial fluid. The wick mechanism 958 serves to enhance coupling to the interstitial space. It could also ensure immediate coupling to the interstitial space without the need for an implantation period. A coupled wick and capsule as shown may ensure that the device works immediately so as to avoid an implantation period.

In one embodiment, the wick 946 is optional.

Once the flush is complete interstitial pressure is exerted on the balloon 956, which, in turn, exerts that pressure on the pressure fluid through the balloon shaft, to the pressure fluid reservoir 914 and then to the pressure sensor 922. In one embodiment, a pressure amplifying system like the one described in FIG. 11 could be integrated into the pressure fluid reservoir 914 for the previously described benefits.

The MCU then processes the signal from the pressure sensor 922 to represent SCIP and communicates the SCIP via Blue Tooth to a device such as a mobile phone.

The data may be processed via simple time series and trending analysis as known to those of skill in the art. In other processing schema, artificial intelligence models (e.g., neural networks) may be trained using SCIP data versus hospitalizations or other abnormalities to inform a predictive model to define when to intervene. In other schema, a threshold algorithm may be employed using physiologic pressure-volume curves relevant to the specific region of anatomy the SCIP measurement is being performed in. For example, the nominal SCIP value (euvolemic state) is negative. Transitions to positive pressures, relative to atmosphere, are associated with massive increases in interstitial volume. Thus, a threshold of 0 mmHg may be an important indicator of acute onset of edema and an important way of determining true physiologic dry weight. Knowing this physiologic relationship, a clinician could use this transition point as an indicator that fluid offloading via, for example, diuretic up-titration should be attempted. It is possible that other areas of the body may be more sensitive to early fluid accumulation than others. Different pressure-volume curves in different organs could lead to earlier or organ specific identification of the degree of fluid overload or edema. Other example areas to place a device to measure interstitial pressure may include the heart, lungs, spleen, the mesentery and kidneys.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.
1 McGeown, M. G. & McGeown, M. G. The fluid compartments of the body. Clin. Manag. Electrolyte Disord. 15-17 (1983) doi:10.1007/978-94-009-6699-4_2.
2 Breslin, J. W. et al. Lymphatic vessel network structure and physiology. Compr. Physiol. 9, (2019).
3 Hu, D. et al. Lymphatic system identification, pathophysiology and therapy in the cardiovascular diseases. J. Mol. Cell. Cardiol. 133, 99-111 (2019).
4 Titze, J. Interstitial fluid homeostasis and pressure: News from the black box. Kidney Int. 84, 869-871 (2013).
5 Stewart, R. H. A Modern View of the Interstitial Space in Health and Disease. Front. Vet. Sci. 7, 1-12 (2020).
6 Wiig, H. & Swartz, M. A. INTERSTITIAL FLUID AND LYMPH FORMATION AND TRANSPORT: PHYSIOLOGICAL REGULATION AND ROLES IN INFLAMMATION AND CANCER. Physiol Rev 92, 1005-1060 (2012).
7 Moore, J. E. & Bertram, C. D. Lymphatic System Flows. Annu. Rev. Fluid Mech. 50, 459-482 (2018).
8 Dupont, M., Mullens, W. & Tang, W. H. W. Impact of systemic venous congestion in heart failure. Curr. Heart Fail. Rep. 8, 233-241 (2011).
9 Damman, K. et al. Increased Central Venous Pressure Is Associated With Impaired Renal Function and Mortality in a Broad Spectrum of Patients With Cardiovascular Disease. J. Am. Coll. Cardiol. 53, 582-588 (2009).
10 Fonarow, G. C., Heywood, J. T., Heidenreich, P. A., Lopatin, M. & Yancy, C. W. Temporal trends in clinical characteristics, treatments, and outcomes for heart failure hospitalizations, 2002 to 2004: findings from Acute Decompensated Heart Failure National Registry (ADHERE). Am. Heart J. 153, 1021-1028 (2007).
11 Gheorghiade, M. et al. Acute heart failure syndromes: Current state and framework for future research. in Circulation vol. 112 3958-3968 (2005).
12 Ganda, A. et al. Venous congestion and endothelial cell activation in acute decompensated heart failure. Current Heart Failure Reports vol. 7 66-74 (2010).
13 Miller, W. L. Fluid volume overload and congestion in heart failure. Circ. Hear. Fail. 9, 1-9 (2016).
14 Szabo, G. & Magyar, Z. Effect of increased systemic venous pressure on lymph pressure and flow.
15 Ratnayake, C. B. B., Escott, A. B. J., Phillips, A. R. J. & Windsor, J. A. The anatomy and physiology of the terminal thoracic duct and ostial valve in health and disease: potential implications for intervention. J. Anat. 233, 1-14 (2018).
16 Cuenoud, H. F., Doern, G. V, Underwood, J. M. & Majno, G. Capillary Leakage in Inflammation A Study by Vascular Labeling. American Journal of Pathology vol. 137 (1990).
17 Claesson-Welsh, L. Vascular permeability—The essentials. Ups. J. Med. Sci. 120, 135-143 (2015).
18 Rothe, C. F. Reflex control of veins and vascular capacitance. Physiol. Rev. 63, 1281-1342 (1983).
19 Miller, W. L. Advances in Heart Failure Fluid Volume Overload and Congestion in Heart Failure Time to Reconsider Pathophysiology and How Volume Is Assessed. I, 1-9 (2016).
20 Chioncel, O. et al. Clinical phenotypes and outcome of patients hospitalized for acute heart failure: the ESC Heart Failure Long-Term Registry. Eur. J. Heart Fail. 19, 1242-1254 (2017).
21 Mullens, W. et al. The use of diuretics in heart failure with congestion—a position statement from the Heart Failure Association of the European Society of Cardiology. Eur. J. Heart Fail. 21, 137-155 (2019).
22 Mentz, R. J. et al. Decongestion Strategies and Renin-Angiotensin-Aldosterone System Activation in Acute Heart Failure. JACC Hear. Fail. 3, 97-107 (2015).
23 Cox, Z. L. & Lenihan, D. J. Loop diuretic resistance in heart failure: Resistance etiology-based strategies to restoring diuretic efficacy. J. Card. Fail. 20, 611-622 (2014).
24 Shah, M. R. & Stevenson, L. W. Searching for evidence: refractory questions in advanced heart failure. J. Card. Fail. 10, 210-218 (2004).
25 Abraham, W. T. & Perl, L. Implantable Hemodynamic Monitoring for Heart Failure Patients. J. Am. Coll. Cardiol. 70, 389-398 (2017).
26 Adamson, P. B. et al. Wireless pulmonary artery pressure monitoring guides management to reduce decompensation in heart failure with preserved ejection fraction. Circ. Hear. Fail. 7, 935-944 (2014).
27 St Jude Medical. CardioMEMS HF System Patient System Guide. 377, 1-6 (2015).
28 Amir, O. et al. Evaluation of remote dielectric sensing (ReDS) technology-guided therapy for decreasing heart failure re-hospitalizations. Int. J. Cardiol. 240, 279-284 (2017).
29 Kelm, D. J. et al. Fluid overload in patients with severe sepsis and septic shock treated with early goal-directed therapy is associated with increased acute need for fluid-related medical interventions and hospital death. Shock 43, 68-73 (2015).
30 Ostermann, M., Straaten, H. M. O. van & Forni, L. G. Fluid overload and acute kidney injury: Cause or consequence? Crit. Care 19, 19-21 (2015).
31 Bouchard, J. et al. Fluid accumulation, survival and recovery of kidney function in critically ill patients with acute kidney injury. Kidney Int. 76, 422-427 (2009).
32 Mitchell, K. H. et al. Volume overload: Prevalence, risk factors, and functional outcome in survivors of septic shock. Ann. Am. Thorac. Soc. 12, 1837-1844 (2015).
33 Douglas, I. S. et al. Fluid Response Evaluation in Sepsis Hypotension and Shock: A Randomized Clinical Trial. Chest 158, 1431-1445 (2020).
34 Sanchez, M. et al. Comparison of Fluid Compartments and Fluid Responsiveness in Septic and Non-Septic Patients. Anaesth. Intensive Care 39, 1022-1029 (2011).
35 SL, M., Xu, J., Kochanek, K. & Arias, E. Mortality in the United States, 2017. NCHS Data Brief 328, (2018).
36 Levey, A. S. et al. Definition and classification of chronic kidney disease: A position statement from Kidney Disease: Improving Global Outcomes (KDIGO). Kidney Int. 67, 2089-2100 (2005).
37 System, U. S. R. D. Chapter 1: Incidence, Prevalence, Patient Characteristics, and Treatment Modalities. 2017 Annu. Data Rep. US Ren. Data 2, (2017).
38 Liyanage, T. et al. Worldwide access to treatment for end-stage kidney disease: a systematic review. Lancet 385, 1975-1982 (2015).
39 Bikbov, B. et al. Global, regional, and national burden of chronic kidney disease, 1990-2017: a systematic analysis for the Global Burden of Disease Study 2017. Lancet 395, 709-733 (2020).
48 U.S. Renal Data System. USRDS 2018 Annual Data Report: Atlas of End-Stage Renal Disease in the United States, Chapter 9: Healthcare Expenditures for Persons with ESRD. 2, 519-528 (2019).
41 Henderson, L. W. Symptomatic hypotension during hemodialysis. Kidney Int. 17, 571-576 (1980).
42 Agarwal, R. Hypervolemia is associated with increased mortality among hemodialysis patients. Hypertension 56, 512-517 (2010).
43 Miguel, S. S. Haemodialysis Dry Weight Assessment: A Literature Review. Ren. Soc. Australas. J. 6, 19-24 (2010).
44 Sinha, A. D. Why assistive technology is needed for probing of dry weight. Blood Purif. 31, 197-202 (2011).
45 Faucon, A. L. et al. Extracellular fluid volume is associated with incident end-stage kidney disease and mortality in patients with chronic kidney disease. Kidney Int. 96, 1020-1029 (2019).
46 Ebah, L. M. et al. Subcutaneous interstitial pressure and volume characteristics in renal impairment associated with edema. Kidney Int. 84, 980-988 (2013).
47 Titze, J. Interstitial fluid homeostasis and pressure: news from the black box. Kidney Int. 84, 869-871 (2013).
48 Zhang, B., Hu, Y. & Pang, Z. Modulating the tumor microenvironment to enhance tumor nanomedicine delivery. Front. Pharmacol. 8, 1-16 (2017).
49 Coates, A. et al. On the receiving end—patient perception of the side-effects of cancer chemotherapy. Eur. J. Cancer Clin. Oncol. 19, 203-208 (1983).
50 Boussios, S., Pentheroudakis, G., Katsanos, K. & Pavlidis, N. Systemic treatment-induced gastrointestinal toxicity: incidence, clinical presentation and management. Ann. Gastroenterol. 25, 106-118 (2012).
51 Steichen, S. D., Caldorera-Moore, M. & Peppas, N. A. A review of current nanoparticle and targeting moieties for the delivery of cancer therapeutics. Eur. J. Pharm. Sci. 48, 416-427 (2013).
52 Jain, R. K. Barriers to Drug Delivery in Solid Tumors. Sci. Am. 271, 58-65 (1994).
53 Jain, R. K. Transport of Molecules in the Tumor Interstitium: A Review. Cancer Res. 47, 3039-3051 (1987).
54 Paskins-Hurlburt, A. J., Hollenberg, N. K. & Abrams, H. L. Tumor perfusion in relation to the rapid growth phase and necrosis: studies on the Walker carcinoma in the rat testicle. Microvasc. Res. 24, 15-24 (1982).
55 Shayan, R., Achen, M. G. & Stacker, S. A. Lymphatic vessels in cancer metastasis: bridging the gaps. Carcinogenesis 27, 1729-1738 (2006).
56 Nandu, V. V & Chaudhari, M. S. Efficacy of Sentinel Lymph Node Biopsy in Detecting Axillary Metastasis in Breast Cancer Using Methylene Blue. Indian J. Surg. Oncol. 8, 109-112 (2017).
57 Heldin, C.-H., Rubin, K., Pietras, K. & Ostman, A. High interstitial fluid pressure—an obstacle in cancer therapy. Nat. Rev. Cancer 4, 806-813 (2004).
58 BOckelmann, L. C. & Schumacher, U. Targeting tumor interstitial fluid pressure: will it yield novel successful therapies for solid tumors? Expert Opin. Ther. Targets 23, 1005-1014 (2019).

1. A method a treating a physiologic condition of a patient comprising:
measuring subcutaneous interstitial pressure (SCIP);
determining whether the SCIP is in a normal range;
conducting a therapy when the SCIP is outside a normal range;
monitoring the SCIP;
discontinuing the therapy when the SCIP is in a normal range.

2. The method according to claim 1, wherein the physiologic condition is one of at least the following conditions: interstitial edema; heart failure; sepsis; kidney disease; and cancer.

3. A method according to claim 1, further comprising:
forming a free fluid pocket in an interstitial space of a patient and wherein measuring the SCIP includes measuring the pressure in the free fluid pocket.

4. A method according to claim 3, wherein the forming a free fluid pocket comprises inserting a compartment in the interstitial space and allowing tissue and capillary ingrowth into said compartment to thereby form the free fluid pocket.

5. A method according to claim 4, wherein the compartment is implanted in a thorax of a patient.

6. A method according to claim 4, wherein the compartment is implanted into the interstitial space for a period of 3-5 weeks.

7. A method according to claim 4, further comprising connecting a pressure sensor into the free fluid pocket.

8. A method according to claim 7, wherein a signal representative of SCIP from the pressure sensor is amplified.

9. A method according to claim 1, further comprising applying a wearable pressure sensing device to the patient and measuring SCIP with the wearable pressure sensing device.

10. A method according to claim 1, further comprising implanting an implantable pressure sensing device in a patient and measuring SCIP with the implantable pressure sensing device.

11. A device for measuring interstitial pressure comprising:
a compartment having small pores,
a pressure conduit connected to the compartment; and,
a pressure sensor connected to the pressure conduit.

12. A device according to claim 11, further comprising a foam positioned on an interior surface of the compartment.

13. A device according to claim 11, wherein the pressure conduit is a needle.

14. A device according to claim 11, wherein the pressure conduit is a balloon lining.

15. A device according to claim 14, wherein the balloon lining is positioned adjacent the foam.

16. A device according to claim 14, wherein the balloon lining has small pores.

17-19. (canceled)

20. A device for measuring interstitial pressure comprising:
a compartment with small pores;
a pressure passage conduit connectable with the compartment,
a pressure sensor connected to the pressure passage.

21. A device according to claim 20, further comprising an amplifier connected to the pressure passage, wherein the amplifier comprises a first fluid chamber and a second fluid chamber connected to each other with a piston system.

22. A device according to claim 21, wherein the piston system comprises a first pressure disc and a second pressure disc wherein a surface area of the first pressure disc is larger than a surface area of a second pressure disc.

23-34. (canceled)
